(19) United States
(12) Patent Application Publication
(10) Pub. No.: US 2026/0248575 A1
(43) Pub. Date: Aug. 27, 2026

(54) GUIDE APPARATUS FOR DELIVERY OF A FLEXIBLE ELONGATE DEVICE AND RELATED METHODS

(71) Applicant: INTUITIVE SURGICAL OPERATIONS, INC., Sunnyvale, CA (US)

(72) Inventor: Salim Dogan Sekercioglu, Mountain View, CA (US)

(21) Appl. No.: 19/548,517

(22) Filed: Feb. 24, 2026

Related U.S. Application Data
(60) Provisional application No. 63/762,375, filed on Feb. 24, 2025.

(51) Int. Cl. A61B 34/37; A61B 34/30; A61M 25/01

(52) U.S. Cl. CPC A61B 34/37; A61B 2034/301; A61M 25/0113

(57) ABSTRACT

A guide and key to support and restrict buckling of a flexible elongate device includes opposing wall portion at least partially defining a channel of the guide. The wall portions form movable regions that move from a rest state to a deformed state to expose the channel more than the rest state. A key for the guide is coupled to an elongate body portion of the flexible elongate device. Movement of the key causes the movable regions to move from the rest state to the deformed state.

### CROSS-REFERENCE TO RELATED APPLICATIONS

This application claims the benefit of priority to U.S. Provisional Application No. 63/762,375, filed Feb. 24, 2025, which is hereby incorporated by reference in its entirety.

### FIELD

Disclosed embodiments relate to flexible elongate devices and, more particularly, to guide systems for flexible elongate devices.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy. In existing systems, at least a portion of the flexible elongate device extending between the patient and a teleoperational manipulator is unsupported, and the flexible nature of the device can cause it to bend, twist, or buckle in an undesirable manner at a point external to the patient's body when force is exerted to insert the instrument into the patient's anatomy. Deformation of the instrument may damage internal components such as optical fiber shape sensors or endoscopic equipment.

### SUMMARY

The following presents a simplified summary of various examples described herein and is not intended to identify key or critical elements or to delineate the scope of the claims.

In accordance with a first example, a medical system is disclosed that includes a guide and a key. The guide includes a first wall portion and a second wall portion, where the first and second wall portions at least partially define a channel along a longitudinal axis of the guide. The first and second wall portions form a series of independently movable regions along the longitudinal axis, each movable region being movable between a rest state and a deformed state that exposes the channel more than the rest state. The key is coupled to an elongate body portion of a flexible elongate device and is configured to move the pairs of first and second protrusions from the rest state to the deformed state as the key moves along the longitudinal axis and the elongate body portion is within the channel.

In some examples, the first wall portion includes a plurality of elastically deformable first protrusions along the longitudinal axis and the second wall portion includes a plurality of elastically deformable second protrusions along the longitudinal axis. Pairs of the first protrusions and second protrusions form each movable region.

In some examples, each movable region encloses the channel in the rest state, the movable regions return to the rest state from the deformed state in the absence of an external force that causes the movable region to be in the deformed state, the first wall portion and the second wall portion are coupled together such that the guide comprises a single piece component, and/or the guide is a single material, such as plastic.

In some examples, the medical system includes the flexible elongate device, the movable regions configured to contain the elongate body portion within the channel in the rest state. In further examples, the flexible elongate device includes a backend mechanism comprising the key and/or the flexible elongate device defines a lumen that extends within the elongate body portion and the key and the key includes a neck portion that extends outside of the channel as the key moves along the longitudinal axis and a body portion that is within the channel as the key moves along the longitudinal axis. In yet further examples, the medical system includes a medical tool configured to be inserted within the lumen of the elongate body portion via the key.

In some examples, the medical system includes a rigid base coupled to the guide. In further examples, the rigid base includes a keyed coupling configured to mount to a manipulator assembly.

In some examples, the medical system includes a curved distal guide coupled to the guide at a distal end thereof, the curved distal guide configured to direct movement of the elongate body portion of the flexible elongate device to an insertion axis transverse to the longitudinal axis of the channel. In further examples, a curvature of the curved distal guide is adjustable.

Any of the above examples can include one or more of the following aspects: the medical system includes a manipulator assembly configured to control movement of a flexible elongate device inserted within the channel, the manipulator assembly including the key; adjacent ones of the movable regions are spaced apart by grooves; the first wall portion and the second wall portion each define a plurality of slits spaced along the longitudinal axis of the guide; the guide has a circular or racetrack cross-section; the longitudinal axis of the guide is a horizontal axis and the horizontal axis is an insertion axis for the flexible elongate device; and/or the first and second protrusions partially overlap in the rest state.

In accordance with a second example, a method is disclosed that includes supporting an elongate body portion of a flexible elongate device against buckling within a channel of a guide, the guide including a first wall portion and a second wall portion at least partially defining the channel, and driving movement of the flexible elongate device within the guide by moving a key coupled to the flexible elongate device longitudinally along the guide, movement of the key causing independently movable regions of the first and second wall portions to move from a rest state to a deformed state to expose the channel.

In some examples, movement of the key elastically deforms aligned first protrusions of the first wall portion and second protrusions of the second wall portion for each movable region, the method includes enclosing the channel with the movable regions in the rest state, and/or the method includes the movable regions returning to the rest state from the deformed state in the absence of an external force that causes the movable region to be in the deformed state.

In some examples, causing the independently movable regions of the first and second wall portions to move from the rest state to the deformed state includes driving a neck portion of the key that extends outside of the channel between the first and second wall portions. In further examples, the method includes inserting a tool into a lumen that extends within the elongate body portion of the flexible elongate device through an opening defined in the key.

In some examples, the method includes controlling movement of the flexible elongate device with a manipulator assembly. In further examples, the method includes mounting a rigid base of the guide to the manipulator assembly with a keyed coupling.

In some examples, driving movement of the flexible elongate device within the guide includes driving movement of the flexible elongate device along a horizontal axis. In further examples, driving movement of the flexible elongate device along the horizontal axis includes driving movement of the flexible elongate device along a horizontal insertion axis.

In some examples, the method includes guiding movement of the flexible elongate device along a curved path through a curved distal guide coupled to the guide at a distal end thereof, such that an insertion axis of the flexible elongate device is transverse to a longitudinal axis of the channel. In further examples, the method includes adjusting a curvature of the curved distal guide prior to guiding movement of the flexible elongate device through the curved distal guide.

In some examples, driving movement of the flexible elongate device within the guide comprises holding the guide stationary.

In accordance with a third example, a guide for a flexible elongate device is disclosed that includes a first wall portion and a second wall portion, the first and second wall portions at least partially defining a channel along a longitudinal axis of the guide. The first and second wall portions define a series of independently movable regions along the longitudinal axis, each movable region being movable between a rest state and a deformed state that exposes the channel more than the rest state

In some examples, the first wall portion includes a plurality of elastically deformable first protrusions along the longitudinal axis and the second wall portion includes a plurality of elastically deformable second protrusions along the longitudinal axis. Pairs of the first protrusions and second protrusions form each movable region.

In some examples, the first and second protrusions are spaced apart a first distance in the deformed state to expose the channel and the first and second protrusions enclose the channel or are spaced apart a second distance smaller than the first distance in the rest state, each movable region encloses the channel in the rest state, and/or the movable regions return to the rest state from the deformed state in the absence of an external force that causes the movable region to be in the deformed state.

In some examples, the first wall portion and the second wall portion are coupled together, such that the guide comprises a single piece component of a single material, and/or adjacent ones of the movable regions are spaced apart by grooves.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4A is a top perspective view of a medical system including a guide and key for a flexible elongate device according to some embodiments.

FIG. 4B is a side plan view of the medical system of FIG. 4A showing the key in a first, proximal position along the guide according to some embodiments.

FIG. 4C is a side plan view of the medical system of FIG. 4A showing the key in a second, distal position along the guide according to some embodiments.

FIG. 4D is a sectional perspective view of the guide and key of FIG. 4A according to some embodiments.

FIG. 4E is a side cross-sectional view of the medical system of FIG. 4A according to some embodiments.

FIG. 4F is a front cross-sectional view of the medical system of FIG. 4A according to some embodiments.

FIG. 4G is a perspective view of the medical system of FIG. 4A showing an installation of the guide and key to a manipulator assembly.

FIG. 5 is a flowchart for operating a medical system according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

A guide for flexible elongate devices is disclosed that enables alternative manipulator architectures by simplifying workflow, manufacturing, and potentially lowering per-procedure costs. The guide can have a single-part construction and be made from a single material. Due to this, the guide can be configured with a desired stiffness/flexibility and be suitable for flexible elongate devices having different diameters and/or different insertion vectors. The guide can be utilized in a horizontal orientation, which can reduce potential complexity and allowing a linear insertion.

The guide has a tubular form with a slot extending longitudinally along a top thereof. The opposing walls on either side of the slot include elastically deformable portions that close the slot at rest and can be resiliently driven apart from one another to expose a channel within the guide. A key for the guide includes a body portion movable longitudinally within the guide and a neck portion that extends through the slot of the guide. Movement of the key causes opposing ones of the deformable portions to move to away from one another to expose the channel. The key is coupled to an elongate body portion of the flexible elongate device and provides a pathway to access a lumen of the flexible elongate device while the elongate body portion is within the guide.

In some examples, the guide is provided as part of a medical device that includes the flexible elongate device and an instrument carriage. The instrument carriage is coupled to the key and configured to direct movement of the key for controlled insertion and retraction of the flexible elongate device.

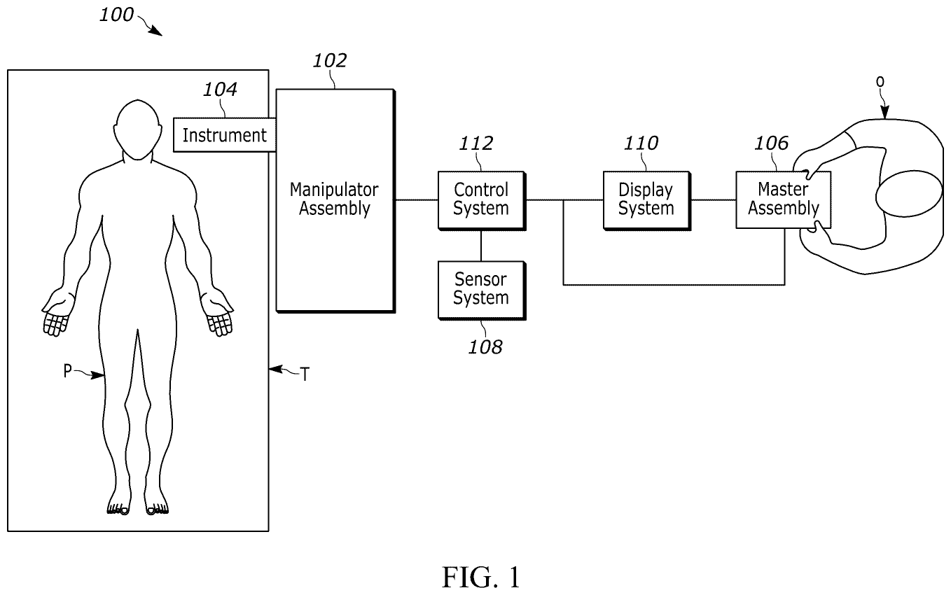

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

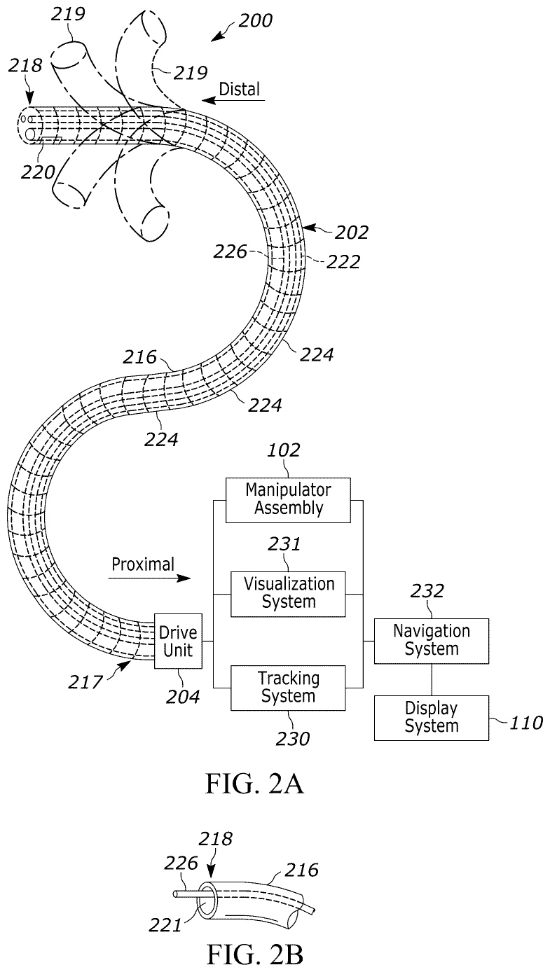

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sept. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sept. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 218. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

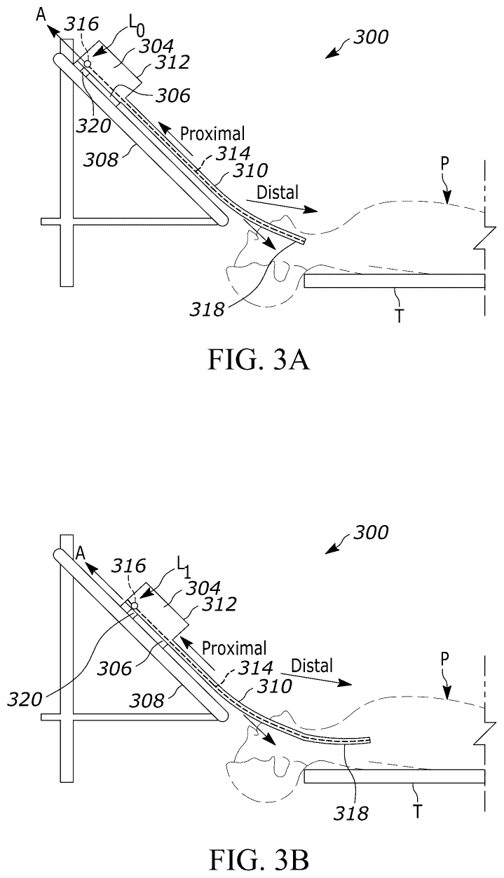

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

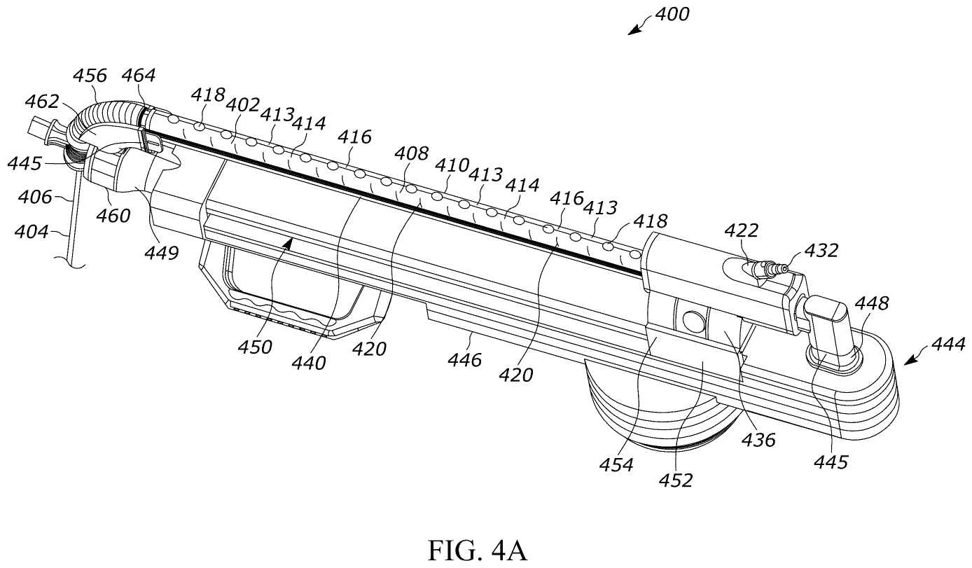

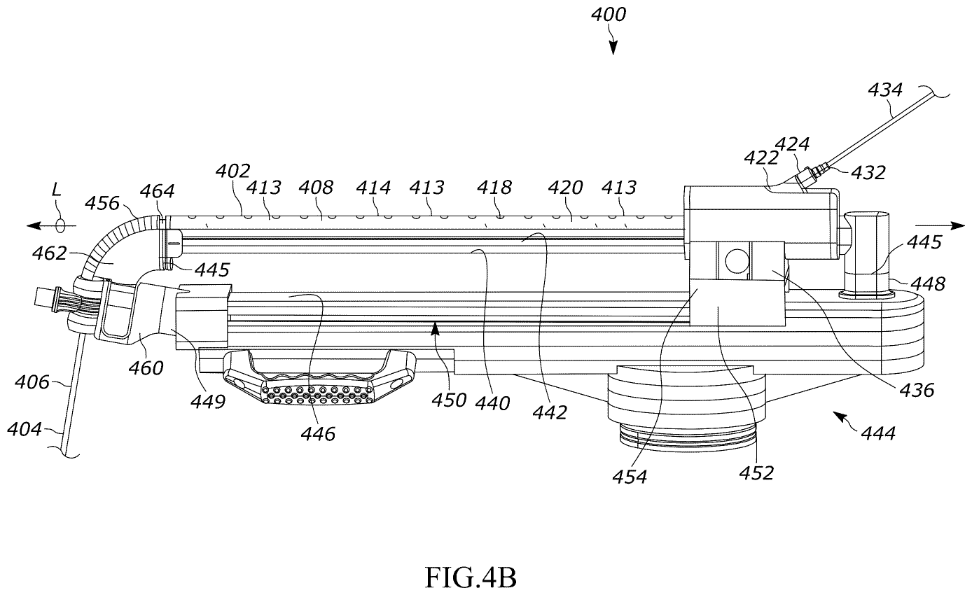

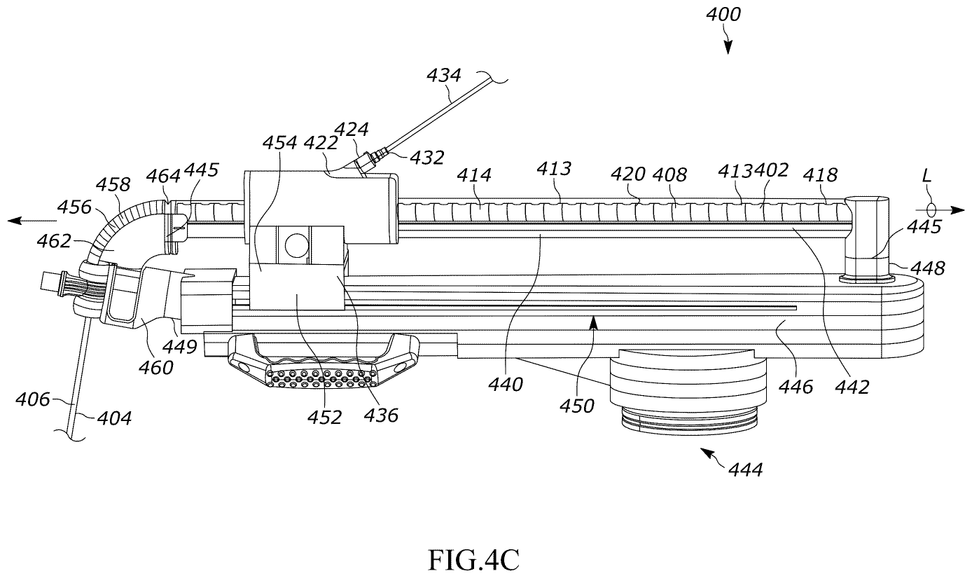

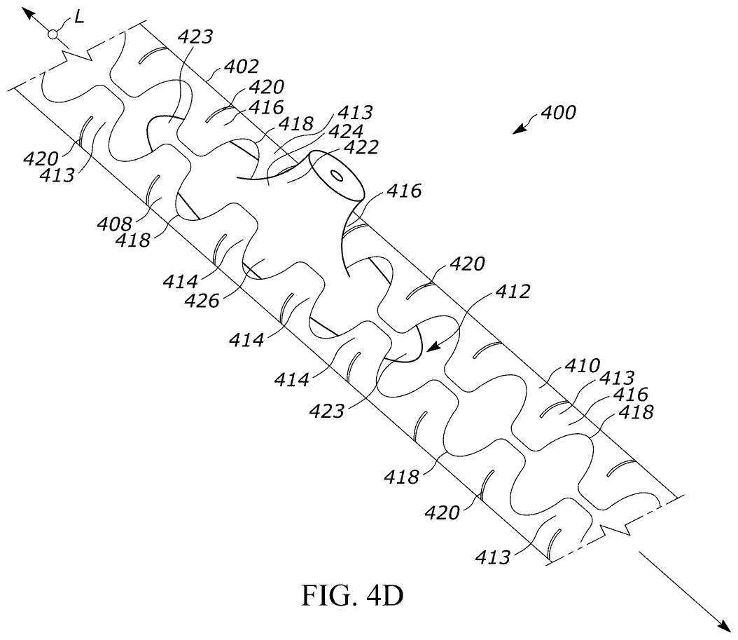

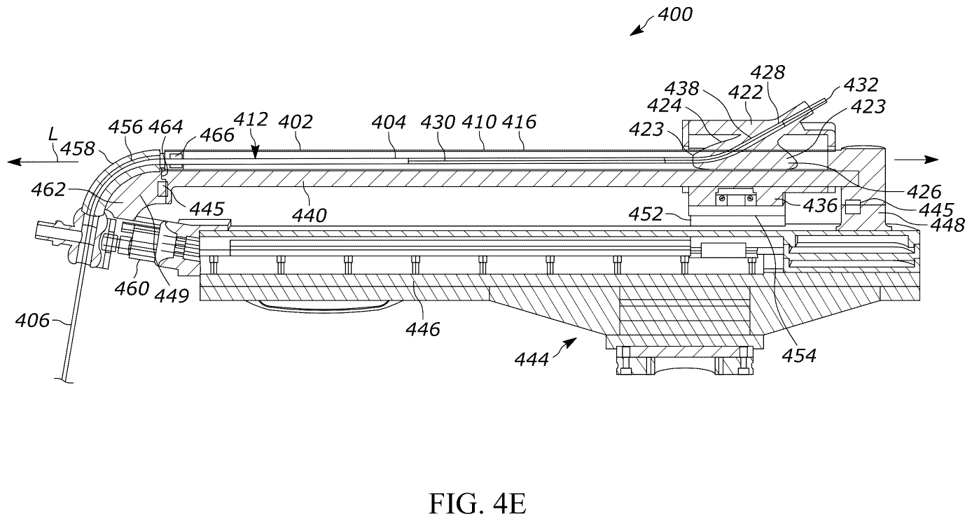

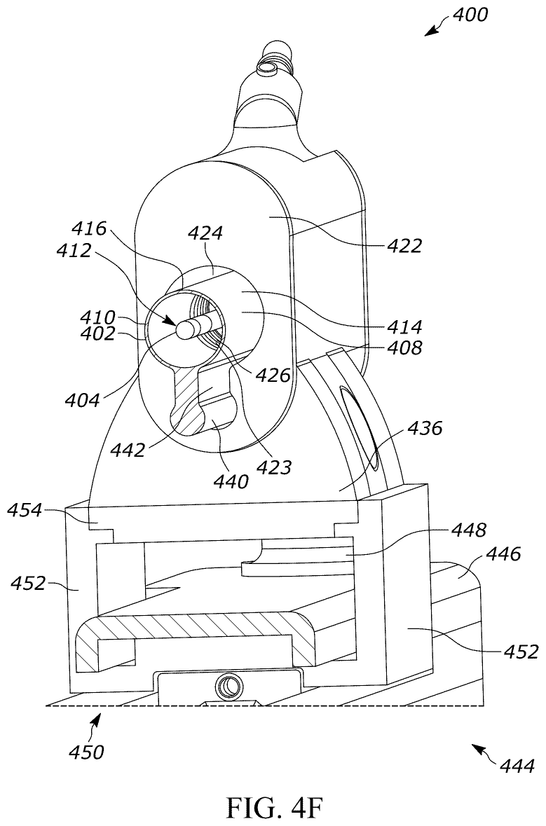

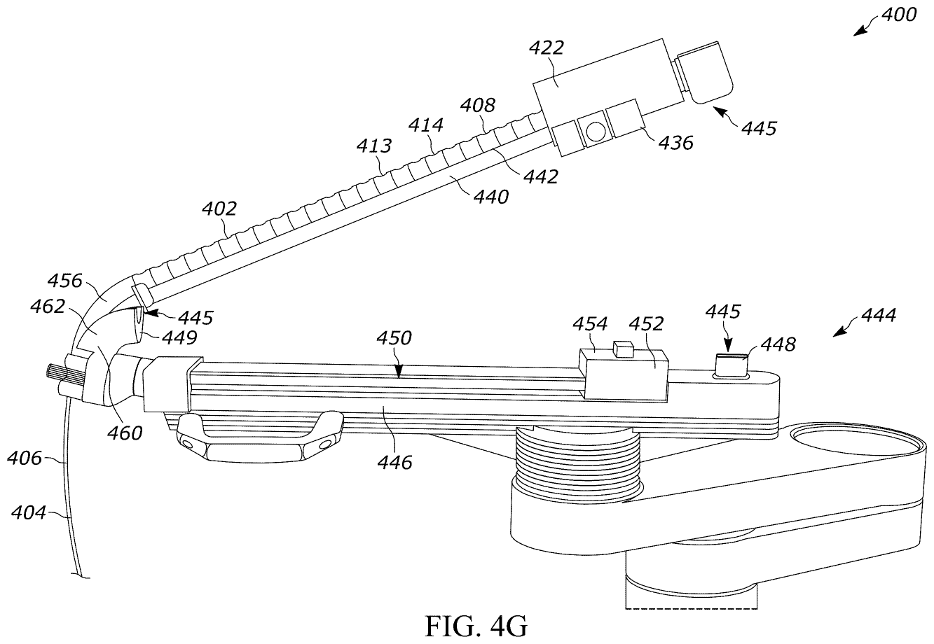

An example medical system 400 is shown in FIGS. 4A-4G. The medical system 400 includes a guide 402 for a flexible elongate device 404 having an elongate body portion 406. The guide 402 is configured to support the flexible elongate device 404 during insertion/retraction and prevent the flexible elongate device 404 from buckling outwardly. This advantageously ensures that a distal end of the flexible elongate device 404 remains in a stable position to allow a distal end to be at a known distance and prevents potential damage to the flexible elongate device 404 during insertion. The flexible elongate device 404 may be used to perform a medical procedure which can include, for example, surgery, biopsy, ablation, illumination, irrigation, suction, or electroporation. The flexible elongate device 404 may be, for example, the medical instrument 104, 304 or elongate device 202.

As shown in FIG. 4A and F, the guide 402 includes a first wall portion 408 and a second wall portion 410 that at least partially define a channel 412 along a longitudinal axis L (FIG. 4B) of the guide 402. The channel 412 can have any suitable cross-sectional shape, such as, for example, a circular shape or a race-track shape. The first and second wall portions 408, 410 form a series of independently movable regions 413 along the longitudinal axis L of the guide 402. Each movable region 413 is movable between a rest state and a deformed state that exposes the channel 412 more than the rest state. In some examples, each movable region 413 can enclose the channel 412 in the rest state and/or return to the rest state from the deformed state in the absence of an external force that causes the movable region 413 to be in the deformed state.

As shown, the first wall portion 408 can include a plurality of elastically deformable first protrusions 414 disposed along the longitudinal axis L and the second wall portion 410 includes a plurality of elastically deformable second protrusions 416 disposed along the longitudinal axis L. In this example, pairs of the first and second protrusions 414, 416 form each movable region 413.

As shown in FIG. 4D, the first protrusions 414 and second protrusions 416 are separated from one another and, in some examples, aligned to form pairs of aligned first and second protrusions 414, 416. Due to the elastic nature of the protrusions 414, 416, each pair of first and second protrusions 414, 416 is independently movable between a deformed state where the first and second protrusions are spaced apart a first distance from one another to expose the channel 412 and a rest state where the first and second protrusions 414, 416 enclose the channel 412 (e.g., abut or partially overlap one another) or are spaced apart a second distance smaller than the first distance. In the latter example, the elongate body portion 406 of the flexible elongate device 404 has a width/diameter larger than the second distance, such that the elongate body portion 406 is held within the channel 412 when the protrusions 414, 416 are in the rest state regardless of whether the protrusions 414, 416 enclose the channel 412 or are spaced apart the second distance.

As shown in FIG. 4A, adjacent ones of movable regions 413 are spaced apart from one another by grooves 418 (e.g., the protrusions 414, 416 are spaced apart from one another by one of the grooves 418). In other examples, the movable regions 413/protrusions 414, 416 can be separated by slits.

In some examples, the guide 402 is a single-piece component where the first wall portion 408 and the second wall portion 410 are coupled together along a bottom of guide 402. With this configuration, the guide 402 is formed from a single wall extending around and defining the channel 412. Additionally, the guide 402 can be made from a single material, such as plastic. In one example, the plastic can be Thermoplastic Polyester Elastomer (TPC-ET), although other suitable materials exist.

As shown in FIGA. 4A-4E, the system 400 further includes a key 422 configured to move along the guide 402. The key 422 is coupled to the elongate body portion 406 of the flexible elongate device 404, such that with the elongate body portion 406 within the channel 412, the key 422 drives movement of the flexible elongate device 404 along the guide 402. The key 422 is configured to move both distally and proximally along the guide 402 to thereby cause insertion of the elongate body portion 406 of the flexible elongate device 404 into a patient and retraction of the elongate body portion 406 of the flexible elongate device 404 from a patient. The key 422 can include tapering (e.g., conical, frusto-conical, etc.) or curved (e.g., conical with a convex or concave curvature) proximal and distal ends 423 (FIG. 4E) to easily move within the channel 412 and cause a gradual deformation of the movable regions 413/protrusions 414, 416.

As the key 422 moves along the longitudinal axis L of the guide 402, the key 422 moves (e.g., elastically deforms) the movable regions 413/pairs of first and second protrusions 414, 416 from the rest state to the deformed state. In some examples, the key 422 includes a neck portion 424 that extends outside of the channel 412 (i.e., between the first and second protrusions 414, 416) and a body portion 426 that is within the channel 412. With this configuration, as the key 422 moves along the longitudinal axis L, the neck portion 424 sequentially moves pairs of the first and second protrusions 414, 416 from the rest state to the deformed state as the body portion 426 moves along the longitudinal axis within the channel 412. The deformed pairs of the first and second protrusions 414, 416 resiliently return to the rest state or at least partially to the rest state after the neck portion 424 is driven therepast.

As stated above, the key 422 is coupled to the elongate body portion 406 of the flexible elongate device 404. Pursuant to this, the key 422 can include a through bore 428 sized to receive the elongate body portion 406 therethrough. In some examples, the elongate body portion 406 defines a lumen or channel 430 that extends within the elongate body portion 406. An opening 432 to the lumen 430 is accessible along the neck portion 424 to insert a medical tool 434 (e.g., medical tool 226) into/through the elongate body portion 406. The tool 434 can include any suitable device, such as a camera or other imaging device, energy treatment device, biopsy device, a gripping device, and so forth.

As shown in FIG. 4B-4F, the system 400 includes a rigid base 440 coupled to the guide 402. The base 440 extends along a bottom of the guide 402 to provide support therefor. In some examples, the base 440 includes longitudinal grooves 442 on opposite sides to provide a track for the key 422/backend mechanism 436 to slide along and maintain a desired orientation relative to the guide 402. As shown in FIG. 4G, the base 440 is configured to couple to a manipulator assembly 444. In some examples, one or more couplings or other locking mechanisms 445 (e.g., magnetic couplings, interlocking components, retaining walls, etc.) between the base 440 and the manipulator assembly 444 include a keyed configuration to ensure a proper alignment and for ease of assembly.

Details of the manipulator assembly 444 are shown in FIGS. 4A-4G. The manipulator assembly 444 includes an insertion stage 446 that extends alongside the guide 402 and the base 440. The insertion stage may be, for example, the insertion stage 308. The insertion stage 446 includes mounts 448, 449 to secure proximal and distal ends of the base 440 and/or guide 402 to the insertion stage 446 and prevent movement of the guide 402 along the longitudinal axis L relative to the insertion stage 446. The mounts 448, 449 can include protrusions or upstanding walls that span the distance between the base 440/guide 402 and the main body of the insertion stage 308. Moreover, the mounts 448, 449 can include one of the couplings 445 to provide a secure and repeatable action to mount the base 440 and/or guide 402 to the insertion stage 308. In some examples, the proximal mount 448 can be an upright support having an upper surface configured to receive the base 440 thereon. The distal mount 449 can have a similar upright support for some applications or can include a ramp for a distal guide, as discussed in more detail below.

As shown, the insertion stage 446 defines a second track 450 or other suitable sliding mechanism extending therealong parallel to the longitudinal axis L of the guide 402. The second track 450 provides an additional sliding connection along with the grooves 442 of the base 440 to allow the key 422 to be easily and repeatably shifted along the longitudinal axis L of the guide 402 when coupled thereto.

In the illustrated example, the flexible elongate device 404 includes a backend mechanism 436 at a proximal end 438 thereof. As shown, the backend mechanism 436 can include the key 422. The backend mechanism 436 includes legs 452 that extend to lateral sides of the insertion stage 446 to engage the track 450 with inwardly extending protrusions. With this configuration, the key 422 is guided and held in an intended orientation relative to the guide 402 by both the engagement with the base 440 and the engagement with the manipulator assembly insertion stage 446.

The backend mechanism 436 can further include an instrument carriage 454. The instrument carriage 454 may be, for example, the instrument carriage 306. The instrument carriage 454 may be a component of a manipulator assembly (e.g., manipulator assembly 102) that couples to the flexible elongate device 404 to control an insertion motion (e.g., motion along an insertion axis) and/or motion of a distal end of the flexible elongate device 404 in multiple directions, such as yaw, pitch, and/or roll. The instrument carriage 454 or insertion stage 446 may include actuators, such as servomotors, that control motion of the instrument carriage 454 along the insertion stage 446. As shown in FIG. 4G, the instrument carriage 454 and the key 422 can be releasably coupled together when the guide 402/base 440 is coupled to the manipulator assembly 444.

In some examples, the longitudinal axis L of the guide 402 is also an insertion axis for the flexible elongate device 404. With this configuration, a patient can be positioned at the end of the guide 402 for the elongate body portion 406 to be inserted into the patient along the insertion axis. In further examples, the longitudinal axis L and the insertion axis can both be horizontal axes (i.e., the guide 402 is positioned horizontally adjacent to the patient). Alternatively, the guide 402 can be disposed at an upward angle relative to the patient for an insertion axis that is angled downwardly to the patient, such as that shown in FIGS. 3A and 3B.

In other examples, the insertion axis for the flexible elongate device 404 is transverse to the longitudinal axis L of the guide 402. In these examples, the system 400 includes a distal guide 456 that guides the elongate body portion 406 along a curved path to a patient. The distal guide 456 can have a fixed curvature or can be adjustable as shown. To be adjustable, the distal guide 456 can have an articulated configuration with interlocking or connected rings (e.g., with wires or other flexible interconnecting structure) that can be manipulated relative to one another along different curvatures. In some examples, the distal guide 456 and the guide 402 can be integral with one another, such as a single piece component. In these examples, the first wall portion 408 and the second wall portion 410 each define a plurality of slits 420 (FIGS. 4A and 4D) spaced along the longitudinal axis L of the guide 402 and spaced from the protrusions 414, 416. The slits 420 can be aligned with the movable regions 413/protrusions 414, 416 as shown, be aligned with the grooves 418, or be offset relative to either. The slits 420 can be utilized to flex the guide 402 and distal guide 456 as needed to perform a particular procedure with a desired curvature for an insertion axis.

With this configuration, the elongate body portion 406 of the flexible elongate device 404 extends through the rings and is guided along the inner surface thereof to the insertion axis. The distal guide 456 can include an outer sleeve 458 to hold the components together. Further, the system 400 can include a mount 460 defining a ramp 462 having a curved surface to receive and support the distal guide 456 during use. The ramp 462 may be utilized to ensure that the distal guide 456 maintains a desired curvature. The mount 460 is coupled to the insertion stage 446 of the manipulator assembly 444 to provide a secure support for the distal end of the guide 402 and the distal guide 456.

To ensure that the guide 402 and distal guide 456 do not move relative to one another and the insertion stage 446 during use, the mount 460 and distal guide 456 couple together with interconnecting members 464 (e.g., tongue-and-groove members, detent members, etc.) that prevent movement along the longitudinal axis L. Further, the distal guide 456 includes an annular plug portion 466 that extends into the guide 402, such that both ends of the guide 402 are rigidly held in place and prevented from movement along the longitudinal axis L.

In some examples, the guide 402 and distal guide 456 can be positioned to extend within a horizontal plane, such that the insertion axis is also horizontal. Alternatively, the guide 402 can be positioned at an upward angle relative to a patient with the distal guide 456 guiding the elongate body portion 406 to an insertion axis transverse to the longitudinal axis L of the guide 402.

Movement of the key 422 and the resulting insertion of the elongate body portion 406 of the flexible elongate device 404 can be hand driven or driven via robotic control. The medical system 400 is suitable for a manipulator assembly, such as the manipulator assembly 102 discussed above.

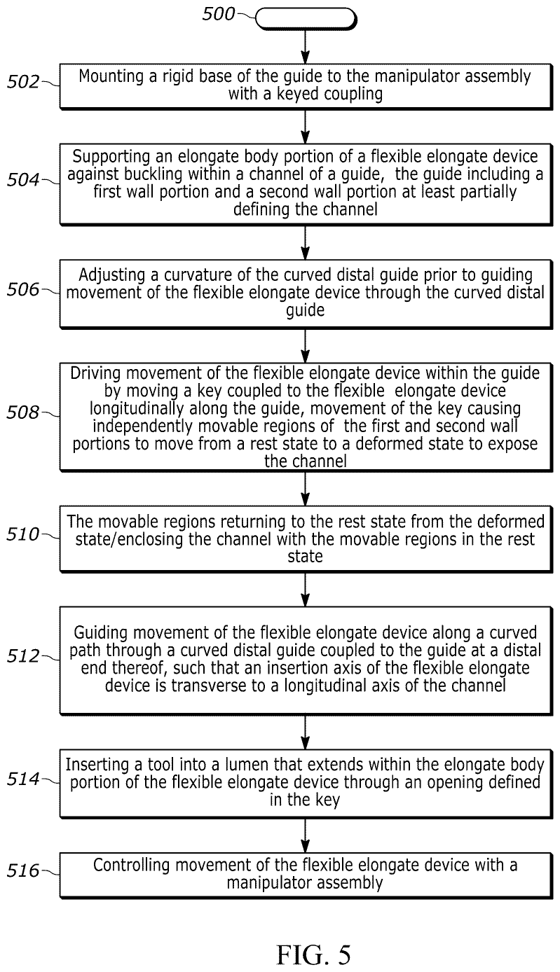

FIG. 5 illustrates a method 500 for operation of a medical system including a flexible elongate device (e.g., the medical system 400 and flexible elongate device 404) according to some embodiments. The method 500 is illustrated as a set of operations or processes 502 through 516. Not all of the illustrated processes may be performed in all embodiments of the method 500. Additionally, one or more processes that are not expressly illustrated in FIG. 5 may be included before, after, in between, or as part of the processes 502 through 516. Processes may also be performed in different orders. In some embodiments, one or more of the processes 502 through 516 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 502 through 516 may be performed by a controller.

In process 502, a rigid base (e.g., rigid base 440) of a guide (e.g., 402) is mounted to a manipulator assembly with a keyed coupling. In process 504, an elongate body portion (e.g., elongate body portion 406) of the flexible elongate device is supported against buckling within a channel (e.g., channel 412) of the guide, the guide including a first wall portion (e.g., first wall portion 408) and a second wall portion (e.g., second wall portion 410) at least partially defining the channel. In process 506, a curvature of a curved distal guide (e.g., distal guide 456) is adjusted.

In process 508, movement of the flexible elongate device is driven within the guide by moving a key (e.g., key 422) coupled to the flexible elongate device longitudinally along the guide, movement of the key causing independently movable regions (e.g., movable regions 413) of the first and second wall portions to move from a rest state to a deformed state to expose the channel. For example, deforming the movable regions can include elastically deforming aligned first protrusions (e.g., first protrusions 414) of the first wall portion and second protrusions (e.g., second protrusions 416) of the second wall portion to expose the channel. In process 510, the movable regions return to the rest state from the deformed state, such as in the absence of an external force that causes the movable region to be in the deformed state and/or the movable regions enclose the channel in the rest state. In process 512, movement of the flexible elongate device is guided along a curved path through the curved distal guide coupled to the guide at a distal end thereof, such that an insertion axis of the flexible elongate device is transverse to a longitudinal axis of the channel.

In process 514, movement of the flexible elongate device is controlled with a manipulator assembly. In process 516, a tool (e.g., tool 434) is inserted into a lumen (e.g., lumen 430) that extends within the elongate body portion of the flexible elongate device through an opening (e.g., 432) defined in the key.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

1. A medical system, comprising:
a guide comprising a first wall portion and a second wall portion, the first and second wall portions at least partially defining a channel along a longitudinal axis of the guide, the first and second wall portions forming a series of independently movable regions along the longitudinal axis, each movable region being movable between a rest state and a deformed state that exposes the channel more than the rest state; and
a key coupled to an elongate body portion of a flexible elongate device, the key configured to move the movable regions from the rest state to the deformed state as the key moves along the longitudinal axis and the elongate body portion is within the channel.

2. The medical system of claim 1, wherein the first wall portion comprises a plurality of elastically deformable first protrusions along the longitudinal axis, the second wall portion comprises a plurality of elastically deformable second protrusions along the longitudinal axis, pairs of the first protrusions and second protrusions forming each movable region.

3. The medical system of claim 1, wherein each movable region encloses the channel in the rest state.

4. The medical system of claim 1, wherein the movable regions return to the rest state from the deformed state in the absence of an external force that causes the movable region to be in the deformed state.

5. The medical system of claim 1, wherein the first wall portion and the second wall portion are coupled together such that the guide comprises a single piece component.

6-7. (canceled)

8. The medical system of claim 1, further comprising the flexible elongate device, the movable regions configured to contain the elongate body portion within the channel in the rest state.

9. (canceled)

10. The medical system of claim 8, wherein the flexible elongate device defines a lumen that extends within the elongate body portion and the key; and the key includes a neck portion that extends outside of the channel as the key moves along the longitudinal axis and a body portion that is within the channel as the key moves along the longitudinal axis.

11. The medical system of claim 10, further comprising a medical tool configured to be inserted within the lumen of the elongate body portion via the key.

12. The medical system of claim 1, further comprising a manipulator assembly configured to control movement of a flexible elongate device inserted within the channel, the manipulator assembly comprising the key.

13. The medical system of claim 1, wherein adjacent ones of the movable regions are spaced apart by grooves.

14. The medical system of claim 1, wherein the first wall portion and the second wall portion each define a plurality of slits spaced along the longitudinal axis of the guide.

15. (canceled)

16. The medical system of claim 1, further comprising a rigid base coupled to the guide, the rigid base comprising a keyed coupling configured to mount to a manipulator assembly.

17. (canceled)

18. The medical system claim 1, wherein the longitudinal axis of the guide is a horizontal axis; and the horizontal axis being an insertion axis for the flexible elongate device.

19. The medical system of claim 1, further comprising a curved distal guide coupled to the guide at a distal end thereof, the curved distal guide configured to direct movement of the elongate body portion of the flexible elongate device to an insertion axis transverse to the longitudinal axis of the channel.

20. (canceled)

21. A method comprising:
supporting an elongate body portion of a flexible elongate device against buckling within a channel of a guide, the guide including a first wall portion and a second wall portion at least partially defining the channel; and
driving movement of the flexible elongate device within the guide by moving a key coupled to the flexible elongate device longitudinally along the guide, movement of the key causing independently movable regions of the first and second wall portions to move from a rest state to a deformed state to expose the channel.

22. The method of claim 21, wherein movement of the key elastically deforms aligned first protrusions of the first wall portion and second protrusions of the second wall portion for each movable region; and further comprising the movable regions returning to the rest state from the deformed state in the absence of an external force that causes the movable region to be in the deformed state.

23. The method of claim 21, further comprising enclosing the channel with the movable regions in the rest state.

24. (canceled)

25. The method of claim 21, wherein causing the independently movable regions of the first and second wall portions to move from the rest state to the deformed state comprises driving a neck portion of the key that extends outside of the channel between the first and second wall portions; and further comprising inserting a tool into a lumen that extends within the elongate body portion of the flexible elongate device through an opening defined in the key.

26-28. (canceled)

29. The method of claim 21, wherein driving movement of the flexible elongate device within the guide comprises driving movement of the flexible elongate device along a horizontal axis.

30. (canceled)

31. The method of claim 21, further comprising guiding movement of the flexible elongate device along a curved path through a curved distal guide coupled to the guide at a distal end thereof, such that an insertion axis of the flexible elongate device is transverse to a longitudinal axis of the channel.

32-40. (canceled)
